(19) United States
(12) Patent Application Publication
(10) Pub. No.: US 2010/0249734 A1
(43) Pub. Date: Sep. 30, 2010

(54) OSTOMY POUCH CONTAINING AND SUPPORTING DEVICE AND SYSTEM

(75) Inventors: Joseph Strang, Mount Horeb, WI (US); David Melnick, Madison, WI (US)

Correspondence Address: Casimir Jones, S.C., 2275 DEMING WAY, SUITE 310, MIDDLETON, WI, 53562, US

(73) Assignee: OSTOMY LIVING, LLC, Madison, WI (US)

(21) Appl. No.: 12/731,714

(22) Filed: Mar. 25, 2010

Related U.S. Application Data
(60) Provisional application No. 61/163,308, filed on Mar. 25, 2009.

(51) Int. Cl. A61F 5/449

(52) U.S. Cl. 604/345

(57) ABSTRACT

The present invention provides a device for containing, positioning, holding, and/or supporting an ostomy pouching system. In particular, the present invention provides a bag comprising one or more shoulder support straps for containing, positioning, holding, and supporting an ostomy pouch over a surgical stoma.

This application claims priority to U.S. Provisional Patent Application Ser. No. 61/163,308 filed Mar. 25, 2009, which is herein incorporated by reference in its entirety.

### FIELD OF THE INVENTION

The present invention provides a device for containing, positioning, holding, and/or supporting an ostomy pouching system. In particular, the present invention provides a bag comprising one or more shoulder support straps for containing, positioning, holding, and supporting an ostomy pouch over a surgical stoma.

### BACKGROUND OF THE INVENTION

Treating some diseases of the digestive or urinary systems can involve removing all or part of a patient's small intestine, colon, rectum or bladder. In these cases, wastes must be rerouted to exit the body of the subject. The rerouting surgery, known as an ostomy, involves diverting a biological system (e.g. colon, ileum, urinary) to a newly created opening in the abdomen wall called a stoma. In an ileostomy, the colon and rectum are surgically removed and the bottom of the small intestine (ileum) is connected to a stoma. In a colostomy, the surgeon removes the rectum and attaches the colon to a stoma. In a urostomy, the ureters, the tubes that carry urine to the bladder, are rerouted to either the small intestine or to the abdominal wall.

An ostomy pouching system (colloquially called a colostomy bag) is a medical prosthetic that provides a means for the collection of waste from a diverted biological system (colon, ileum, urinary) as it exists the stoma. Pouching systems usually consist of a mounting plate, commonly called a wafer, and a collection pouch that is mechanically attached in an airtight seal. Ostomy pouching systems collect waste that is output from a stoma. The pouching system allows the stoma to drain into a sealed collection pouch, while protecting the surrounding skin from contamination. Ostomy pouching systems are air- and water-tight (i.e. fluid-tight) and allow the wearer to lead an active lifestyle. Pouching systems come in one- and two-piece designs. In the one-piece design the ostomy pouch interacts directly with the stoma and surrounding area, forming a seal over the stoma. In the two-piece design, a wafer is attached to the skin, often with a “peel and stick” adhesive backing, making a fluid tight seal around the stoma, and providing a reusable attachment site for the ostomy pouch. The selection of systems varies greatly between individuals and is often based on personal preference and lifestyle.

The weight of an ostomy pouch can place tension on the ostomy pouching system connection (e.g. between pouch and wafer, between wafer and patient, between pouch and subject, etc.). The weight of the pouch can also cause discomfort, irritation, soreness and/or bruising to the patient.

### SUMMARY

In some embodiments, the present invention provides a device for positioning an ostomy pouch on a subject with a surgical stoma comprising providing: (a) an ostomy pouch placement component, and (b) one or more shoulder straps, wherein both ends of each of the one or more shoulder straps are attached to the ostomy pouch placement component, wherein the device is configured to position the ostomy pouch on the subject for proper interaction with the surgical stoma. In some embodiments, the device comprises one strap. In some embodiments, the device comprises two straps. In some embodiments, the ends of the straps are connected to the upper corners of the ostomy pouch placement component. In some embodiments, one or more straps are detachable from and re-attachable to ostomy pouch placement component at one or both end of the straps. In some embodiments, the ostomy pouch placement component comprises a bag. In some embodiments, the ostomy pouch placement component comprises a sleeve. In some embodiments, the device is configured for holding an ostomy pouch in position for proper interaction with a surgical stoma. In some embodiments, the device is configured for supporting a portion of the mass of an ostomy pouch. In some embodiments, the device is configured for supporting the entire mass of an ostomy pouch. In some embodiments, the surgical stoma is the result of a colostomy on the subject. In some embodiments, the surgical stoma is the result of an ileostomy on the subject. In some embodiments, the surgical stoma is the result of a urostomy on the subject. In some embodiments, the proper interaction between the ostomy pouch and the surgical stoma comprises positioning the opening of the ostomy pouch over the stoma. In some embodiments, the proper interaction between the ostomy pouch and the surgical stoma comprises attachment of the opening of the ostomy pouch over the stoma. In some embodiments, the proper interaction between the ostomy pouch and the surgical stoma comprises formation of a fluid-tight seal between of the opening of the ostomy pouch and the stoma. In some embodiments, the proper interaction between the ostomy pouch and the surgical stoma comprises positioning the opening of the ostomy pouch over an ostomy wafer positioned over the stoma. In some embodiments, the proper interaction between the ostomy pouch and the surgical stoma comprises attachment of the opening of the ostomy pouch to an ostomy wafer positioned over the stoma. In some embodiments, the proper interaction between the ostomy pouch and the surgical stoma comprises formation of a fluid-tight seal between the opening of the ostomy pouch and an ostomy wafer positioned over the stoma.

### BRIEF DESCRIPTION OF THE DRAWINGS

The foregoing summary and detailed description is better understood when read in conjunction with the accompanying drawings which are included by way of example and not by way of limitation.

FIG. 1 shows a schematic of a device of the present invention assembled from a front piece and a back piece.

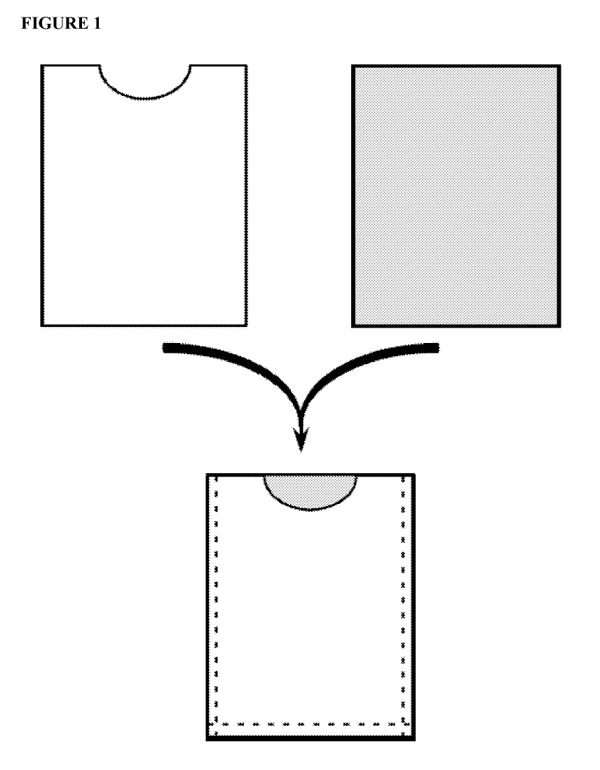

FIG. 2 shows a schematic of a device of the present invention: (a) device only and (b) device containing an ostomy pouch.

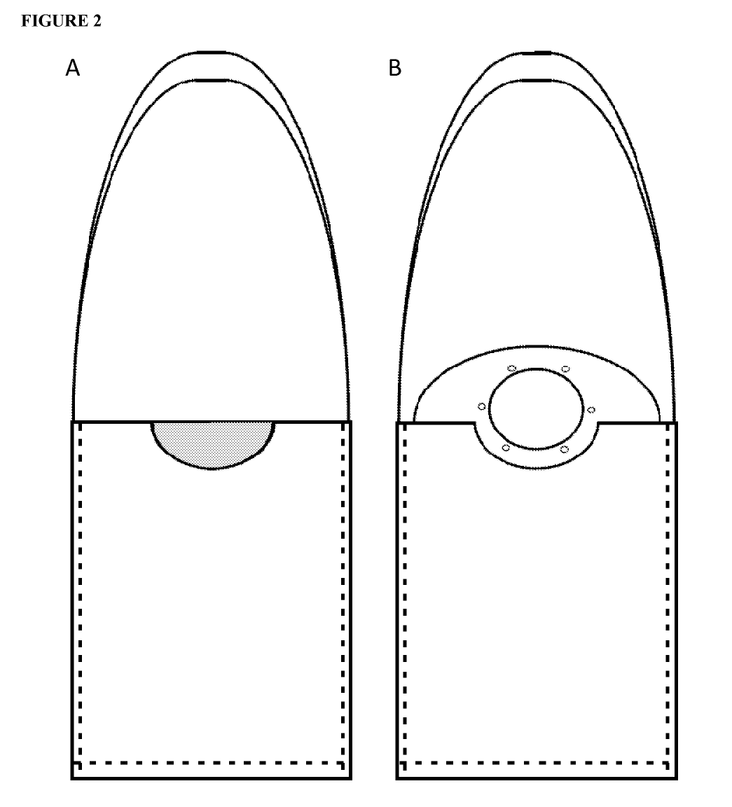

FIG. 3 shows a schematic of a device of the present invention in a two-strap configuration.

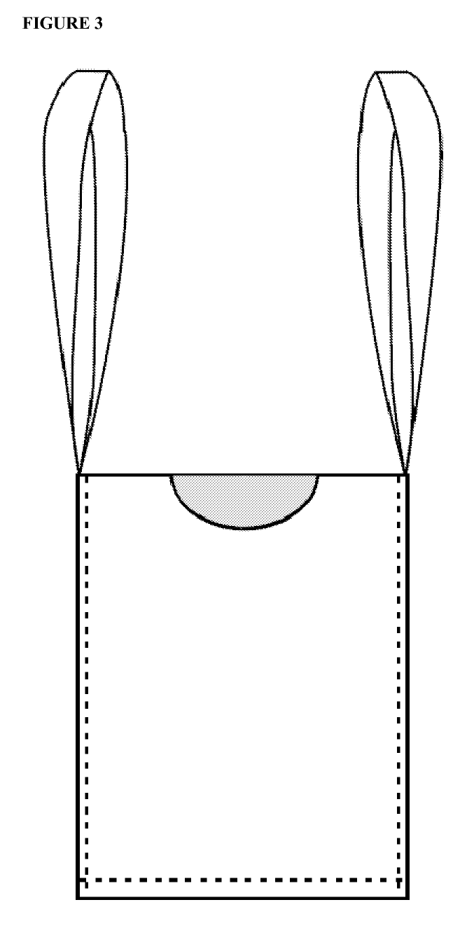

### DETAILED DESCRIPTION OF EMBODIMENTS

The present invention provides a device for containing, positioning, holding, and/or supporting an ostomy pouching system. In particular, the present invention provides a bag comprising one or more shoulder support straps for containing, positioning, holding, and supporting an ostomy pouch over a surgical stoma.

In some embodiments, the present invention provides a component (i.e., housing component) configured to house an ostomy pouch. In some embodiments, this component is one or more of a bag, sack, satchel, pouch, pocket, sleeve, carrier, purse, case, container, etc. In some embodiments, the component is made of any suitable material including, but not limited to fabric or cloth (e.g. wool, cotton, silk, hemp, flax, jute, modal, tencel, polyester, aramid, acrylic, nylon, spandex, olefin fiber, ingeo, etc.), plastic (e.g. rubber, polystyrene, polyvinyl chloride, polyamide, synthetic rubber, polymethyl methacrylate (PMMA), low density polyethylene (PE), high density PE, polypropylene (PP), polyurethane, polyepoxide, polyethylene terephthalate (PET or PETE), polyethylene terephthalate (PET or PETE), polycarbonate, etc.), any derivatives thereof, or any combinations thereof. In some embodiments, the component comprises a front piece and a rear piece, attached on their peripheries. In some embodiments, the component comprises a front piece and a rear piece connected by two side pieces and a bottom piece. In some embodiments, the component has at least one open end. In some embodiments, the component has at least one sealable end. In some embodiments, the component is of an appropriate size to accommodate an ostomy pouch within, while holding the ostomy pouch securely and not allowing the ostomy pouch to shift within the component. In some embodiments, the component is sized appropriately for the specific size of the ostomy pouch contained within (e.g. 5 inch, 6, inch, 7 inch, 8 inch, 9 inch, 10 inch, 11 inch, 12 inch, 13 inch, 14 inch, 15, inch, 16 inch, etc.) In some embodiments, the component is configured for standard pouches, mini-pouches, both, or either. In some embodiments, the component is configured to accommodate and secure a range of ostomy pouch sizes. In some embodiments, the component is sized appropriately for the specific size of the collar or opening of the ostomy pouch contained within (e.g. 0.5 inches . . . 0.75 inches . . . 1.0 inches . . . 1.25 inches . . . 1.5 inches . . . 1.75 inches . . . 2.0 inches . . . 2.25 inches . . . 2.5 inches . . . 2.75 inches . . . 3.0 inches . . . 3.25 inches . . . ). In some embodiments, the component is configured to accommodate and secure a range of ostomy pouch collar sizes. In some embodiments, the component is configured for a disposable ostomy pouch, reusable ostomy pouch, or either. In some embodiments, the component is configured for a one-piece ostomy pouch, two-piece ostomy, pouch, or either. In some embodiments, the component is configured for any suitable stoma-attachment mechanism, including but not limited to one-piece ostomy bags that attach directly to a stoma, peal-and-stick wafer attachment, press-on (“Tupperware” type) attachment, turning locking rings, and “sticky” attachment. In some embodiments, the component is configured for open-end pouches, closed-end pouches, or either.

In some embodiments, the present invention provides one or more components (i.e., support component) (e.g., straps) for supporting the component configured to house an ostomy pouch. In some embodiments, the support component is made of any suitable material including, but not limited to fabric or cloth (e.g. wool, cotton, silk, hemp, flax, jute, modal, tencel, polyester, aramid, acrylic, nylon, spandex, olefin fiber, ingeo, etc.), plastic (e.g. rubber, polystyrene, polyvinyl chloride, polyamide, synthetic rubber, polymethyl methacrylate (PMMA), low density polyethylene (PE), high density PE, polypropylene (PP), polyurethane, polyepoxide, polyethylene terephthalate (PET or PETE), polyethylene terephthalate (PET or PETE), polycarbonate, etc.), any derivatives thereof, or any combinations thereof. In some embodiments, the support component is any appropriate length, width, size or design. In some embodiments, the support component has a length of 1 foot . . . 2 feet . . . 3 feet . . . 4 feet . . . 5 feet . . . 6 feet . . . 7 feet . . . 8 feet . . . 9 feet . . . 10 feet, or any appropriate length. In some embodiments, the support component has a width of 0.25 inches . . . 0.5 inches . . . 0.75 inches . . . 1.0 inches . . . 1.25 inches . . . 1.5 inches . . . 1.75 inches . . . 2.0 inches . . . 2.25 inches . . . 2.5 inches . . . 2.75 inches . . . 3.0 inches . . . 3.25 inches, or any appropriate width. In some embodiments, the support component is attached to housing component. In some embodiments, the support component is releasably attached to the housing component. In some embodiments, one end of a support component is releasably attached to the housing component. In some embodiments, one end of a support component is attached in a fixed-manner to the housing component. In some embodiments, one end of a support component is releasably attached to the housing component and one end is attached in a fixed-manner to the housing component. In some embodiments, the support component supports the housing component using one or more of straps, belts, fasteners, bands, braces, etc. In some embodiments, a support component comprises one or more straps. In some embodiments, one or more straps of the present invention extend from the housing component over the shoulder or shoulders of a subject. In some embodiments, the device is worn from the shoulders of a subject, eliminating restrictions around the waist area that can cause irritation and discomfort. In some embodiments, the support component is configured to engage the subject in any manner that holds and secures the ostomy pouch at the stoma. In some embodiments, straps may wrap around a subject in any suitable manner (e.g. over one shoulder, over both shoulders, around the neck, around a leg, around the waist, any combination thereof, etc.).

The devices of the present invention find use in relieving physical stress placed on the stoma of a subject by an ostomy pouching system. In some embodiments, the present invention relieves physical stress placed on a subject by an ostomy pouching system. In some embodiments, the present invention provides assistance in changing and/or replacing an ostomy pouch. In some embodiments, the present invention relieves emotional stress of a subject by freeing them from worry about their ostomy pouching system. In some embodiments, the present invention provides improved self image as it frees the subject from worries about their ostomy pouching systems. In some embodiments, the present invention can be worn by the patient at any time after surgery, allowing the patient mobility without having to carry the ostomy pouch by hand or by another method. In some embodiments, the present invention provides improved access to the ostomy pouching system at the same time providing an improved system for retaining and support. The present invention can be worn under the subject's normal outer garments and can be imperceptible. In some embodiments, the present invention can provide support of an ostomy pouch for a subject of any size, age, height, weight, or dimensions.

We claim:

1. A device for positioning an ostomy pouch on a subject with a surgical stoma comprising providing: (a) a housing component that substantially surrounds an ostomy pouch; and (b) one or more shoulder straps, wherein both ends of each of said one or more shoulder straps are attached to said housing component; wherein said device is configured to position said ostomy pouch on said subject for proper interaction with a surgical stoma.

2. The device of claim 1, wherein said one or more straps is one strap.

3. The device of claim 2, wherein said ends of said strap are connected to the upper corners of said housing component.

4. The device of claim 1, wherein said one or more straps is two straps.

5. The device of claim 1, wherein one or more ends of said one or more straps are detachable from and re-attachable to said housing component.

6. The device of claim 1, wherein said housing component comprises a bag.

7. The device of claim 1, wherein said housing component comprises a sleeve.

8. The device of claim 1, wherein said device is further configured for holding said ostomy pouch in position for proper interaction with said surgical stoma.

9. The device of claim 1, wherein said device is further configured for supporting a portion of the mass of said ostomy pouch.

10. The device of claim 9, wherein said portion of the mass of said ostomy pouch comprises the entire mass of said ostomy pouch.

11. The device of claim 1, wherein said surgical stoma is the result of a colostomy on said subject.

12. The device of claim 1, wherein said surgical stoma is the result of an ileostomy on said subject.

13. The device of claim 1, wherein said surgical stoma is the result of a urostomy on said subject.

14. The device of claim 1, wherein said proper interaction with said surgical stoma comprises positioning the opening of said ostomy pouch over said stoma.

15. The device of claim 14, wherein said proper interaction comprises attachment of said opening of said ostomy pouch over said stoma.

16. The device of claim 15, wherein said attachment further comprises formation of a fluid-tight seal between said ostomy pouch and said stoma.

17. The device of claim 1, wherein said proper interaction with said surgical stoma comprises positioning the opening of said ostomy pouch over an ostomy wafer positioned over said stoma.

18. The device of claim 17, wherein said proper interaction comprises attachment of said the opening of said ostomy pouch to said ostomy wafer.

19. The device of claim 18, wherein said attachment further comprises formation of a fluid-tight seal between said ostomy pouch and said ostomy wafer.
